(19) United States
(12) Patent Application Publication
(10) Pub. No.: US 2010/0152498 A1
(43) Pub. Date: Jun. 17, 2010

(54) METHODS FOR IMPROVING SYNGAS-TO-ETHANOL CATALYST SELECTIVITY

(75) Inventors: Karl KHARAS, Louisville, CO (US); Esther M. Wilcox, Longmont, CO (US); Heinz Juergen Robota, Arvada, CO (US)

Correspondence Address: Range Fuels, Inc.;Attn: Ryan O'Connor, 11101 West 120th Ave. Suite 200, Broomfield, CO, 80021, US

(73) Assignee: Range Fuels, Inc., Broomfield, CO (US)

(21) Appl. No.: 12/637,078

(22) Filed: Dec. 14, 2009

Related U.S. Application Data
(60) Provisional application No. 61/122,833, filed on Dec. 16, 2008.

(51) Int. Cl. C07C 29/00; B01J 27/051; B01J 23/75

(52) U.S. Cl. 568/840; 502/220

(57) ABSTRACT

The present invention provides methods to increase yields and selectivities to particular alcohols, such as ethanol, during alcohol synthesis from syngas. In some embodiments, a starting catalyst can be activated by contacting with a gas stream under certain preferred activation temperatures, pressures, and compositions.

### PRIORITY DATA

This patent application claims priority under 35 U.S.C. §120 from U.S. Provisional Patent Application No. 61/122,833 for “METHODS FOR IMPROVING SYNGAS-TO-ETHANOL CATALYST SELECTIVITY,” filed Dec. 16, 2008, the disclosure of which is hereby incorporated by reference herein for all purposes.

### FIELD OF THE INVENTION

The present invention generally relates to the field of catalysts and methods for producing alcohols from synthesis gas.

### BACKGROUND OF THE INVENTION

Synthesis gas (hereinafter referred to as syngas) is a mixture of hydrogen (H₂) and carbon monoxide (CO). Syngas can be produced, in principle, from virtually any material containing carbon. Carbonaceous materials commonly include fossil resources such as natural gas, petroleum, coal, and lignite; and renewable resources such as lignocellulosic biomass and various carbon-rich waste materials. It is preferable to utilize a renewable resource to produce syngas because of the rising economic, environmental, and social costs associated with fossil resources.

There exist a variety of conversion technologies to turn these feedstocks into syngas. Conversion approaches can utilize a combination of one or more steps comprising gasification, pyrolysis, steam reforming, and/or partial oxidation of a carbon-containing feedstock.

Syngas is a platform intermediate in the chemical and biorefining industries and has a vast number of uses. Syngas can be directly combusted to produce heat and power. Syngas can also be converted into alkanes, olefins, oxygenates, and alcohols such as methanol, ethanol, and higher alcohols. These chemicals can be blended into, or used directly as, diesel fuel, gasoline, and other liquid fuels.

Since the 1920s it has been known that mixtures of methanol, ethanol, and other linear alcohols can be obtained by reacting syngas over certain catalysts (Fischer and Tropsch, Brennst.-Chem. 7:97, 1926). Later, Dow Chemical and Union Carbide jointly developed a sulfided mixed-alcohol catalyst based on MoS₂ (Phillips et al., National Renewable Energy Laboratory TP-510-41168, April 2007). U.S. Pat. No. 4,752,623 (Stevens and Conway), originally assigned to Dow Chemical, discloses a cobalt-molybdenum-sulfide catalyst for producing mixed alcohols from syngas. However, known catalysts used for the conversion of syngas to alcohols can have limited yields and selectivities to particular alcohols (such as ethanol).

What are therefore needed are methods to increase yields and selectivities to particular C₁-C₄ alcohols. Preferred methods would produce higher carbon-atom selectivities to ethanol compared to methanol, for example. It is preferable that any improved methods, and apparatus to carry out the methods, not substantially introduce incremental operating or capital costs.

### SUMMARY OF THE INVENTION

The present invention addresses the aforementioned needs in the art.

In some embodiments, this invention provides a method of activating a starting catalyst, the method comprising contacting the starting catalyst with a gas phase comprising syngas, under conditions comprising an activation temperature selected from about 200-350° C. and an activation pressure selected from about 25-85 atm, thereby producing an activated catalyst. This invention also describes and includes activated catalysts produced according to these methods.

In particular embodiments, the activation pressure is selected from about 50-75 atm, such as about 60-65 atm. In particular embodiments, the activation temperature is selected from about 250-325° C., such as about 275-300° C.

The activated catalyst can include cobalt, molybdenum, sulfur, and potassium, in some embodiments.

Some methods of the invention further include converting syngas to at least one C₁-C₄ alcohol (e.g., methanol and/or ethanol) over the activated catalyst.

In some embodiments, activating and the converting are conducted in the same vessel. In some embodiments, the activation temperature is about the same as a temperature (e.g., an average or apparent temperature) employed in the converting step. Or, the activation temperature can be lower than the temperature employed in the converting step. In some embodiments, the activation pressure is about the same as a pressure (e.g., an average or apparent pressure) employed in the converting step. Or, the activation pressure can be lower than the pressure employed in the converting step. For example, the activation pressure can be at least 10 atm, or at least 20 atm, lower than the pressure employed in the converting step.

In some variations of the invention, syngas can be converted into methanol and ethanol by a method comprising:

(a) providing a starting catalyst composition;

(b) contacting the starting catalyst with a gas phase comprising syngas, at an activation temperature selected from about 200-350° C. and an activation pressure selected from about 25-85 atm, thereby producing an activated catalyst; and

(c) converting an amount of the syngas to at least some methanol and ethanol over the activated catalyst at an effective process temperature and pressure;

wherein the carbon-atom selectivity ratio of ethanol to methanol, calculated at a CO conversion in accordance with step (c), is at least 0.75.

In various embodiments, the carbon-atom selectivity ratio of ethanol to methanol is at least 1, 1.1, 1.2, or more. In certain embodiments, the carbon-atom selectivity ratio of ethanol to methanol is about 1.25. In some embodiments, the CO conversion is at least 20%, 25%, or more.

The carbon-atom selectivity ratio of ethanol to methanol is preferably higher than the carbon-atom selectivity ratio produced in a comparative method wherein the contacting (for catalyst activation) is carried out at an activation temperature outside the range of 200-350° C. and/or an activation pressure outside the range of 25-85 atm. This comparative method includes converting syngas to methanol and ethanol over the activated catalyst at a suitable process temperature and pressure to provide for substantially the same CO conversion.

Certain embodiments provide a method of activating a Co/Mo/S/K catalyst, the method comprising contacting the Co/Mo/S/K catalyst with a gas phase comprising syngas, at an activation temperature selected from about 275-300° C. and an activation pressure selected from about 60-65 atm, thereby producing an activated catalyst effective for conversion to methanol and ethanol when in the presence of syngas at suitable alcohol-synthesis conditions, wherein at the suitable alcohol-synthesis conditions, the carbon-atom selectivity ratio of ethanol to methanol is at least 1, 1.1, 1.2, or greater.

### BRIEF DESCRIPTION OF THE FIGURE

FIG. 1 depicts catalyst-selectivity data generated in some embodiments of the present invention.

### DETAILED DESCRIPTION OF EMBODIMENTS OF THE INVENTION

This description will enable one skilled in the art to make and use the invention, and it describes several embodiments, adaptations, variations, alternatives, and uses of the invention, including what is presently believed to be the best mode of carrying out the invention.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in their respective testing measurements.

As used herein, “C₁-C₄ alcohols” means one or more alcohols selected from methanol, ethanol, propanol, and butanol, including all known isomers of such compounds. Product selectivities are calculated herein on a carbon-atom basis. “Carbon-atom selectivity” means the ratio of the moles of a specific product to the total moles of all products, scaled by the number of carbon atoms in the species. This definition accounts for the mole-number change due to reaction. The selectivity Sⱼ to general product species Cₓ_{ⱼ}H_{y}_{ⱼ}O_{z}_{ⱼ} is

$${S}_{j} = {x}_{j} {F}_{j} / {\sum }_{i} {x}_{i} {F}_{i}$$

wherein Fⱼ is the molar flow rate of species j which contains xⱼ carbon atoms. The summation is over all carbon-containing species (Cₓ_{ᵢ}H_{y}_{ᵢ}O_{z}_{ᵢ}) produced in the reaction.

The present invention will now be described by reference to the following detailed description and accompanying drawings which characterize and illustrate some preferred embodiments for producing ethanol. This description by no means limits the scope and spirit of the present invention. For example, preferred embodiments relate to Co/Mo/S catalyst compositions, but the invention is by no means so limited.

The present invention is premised, at least in part, on the realization that higher carbon-atom selectivities to ethanol, compared to methanol, can be achieved by activating a suitable catalyst using certain non-obvious conditions. One such suitable catalyst comprises cobalt, molybdenum, sulfur, and potassium.

In some embodiments, a fresh catalyst is contacted with a gas phase comprising syngas, under conditions comprising an activation temperature and an activation pressure, and for a suitable amount of time, thereby producing an activated catalyst for the conversion of syngas to alcohol. An “activated catalyst” can be a catalyst that is activated for the first time, or a catalyst that is re-activated following at least some deactivation. In some variations, the present invention provides methods for activating base-promoted Co/Mo/S catalysts for production of C₁-C₄ alcohols from syngas.

In some embodiments, a starting catalyst is activated in the presence of a gas phase comprising syngas, at an activation temperature selected from about 200-350° C. and an activation pressure selected from about 20-85 atm.

The gas composition for activation preferably includes both hydrogen and carbon monoxide (the components of syngas). The presence of syngas can encourage catalyst activation or re-activation. Activation can lead to higher catalyst activity, better product selectivities, or both of these enhancements. In some embodiments, the activation gas composition further includes carbon dioxide, methane, ethane, ethylene, and so on.

Inert gases are included in the gas composition for activation, in some embodiments. An inert gas can be selected from the group consisting of He, Ne, Ar, Kr, Xe, Rn, or mixtures of any of these. In some embodiments, N₂ can be employed as the inert gas or as part of a mixture of inert gases. Ar can be used as an inert tracer gas, for mass-balancing purposes.

Inert gases, in some variations, can provide for “inert-gas annealing” as described and claimed in copending U.S. Patent App. No. 61/100,024, filed Sep. 25, 2008, the assignee of which is the same assignee of the present application, and which application is hereby incorporated by reference herein.

In some embodiments, the activated catalyst is then subjected to a process (i.e., post-activation) temperature higher than the activation temperature, in the presence of syngas, to convert some of the syngas into alcohols. To illustrate, an exemplary activation temperature is about 270-290° C.; an exemplary process temperature is about 310-330° C.

The activation pressure can be the same as the pressure used to convert syngas into alcohols, or different pressures can be employed. Generally, it is preferred to use an activation pressure that is no greater than the process pressure. In some embodiments, the activation pressure is much lower than the process pressure. For example, the process pressure can be at least 10 atm, or at least 20 atm, higher than the activation pressure.

The activation temperature, activation pressure, activation temperature, and process temperature can be independently selected. On the other hand, it is recognized that in some embodiments, catalyst activation at reduced pressures can enable similar or better catalyst performance at process pressures lower than would have otherwise been necessary. An ability to operate at lower pressure with the same, or improved, carbon selectivity to ethanol (or another desired product) would tend to decrease compression costs and improve the fraction of feedstock carbon that converts into the desired product.

The amount of time that is preferred for the activation step is not regarded as critical to the invention. The times will generally be a function of temperature, pressure, starting compositions, and desired activity, as will be recognized by a person of ordinary skill in the art. In various embodiments, activation times can be less than 1 hour, between about 1-10 hours, or between about 10-50 hours. In certain embodiments, activation times are at least 5 hours.

Some embodiments of the present invention provide for convenient in situ activation of a catalyst composition contained in a reactor. A starting catalyst composition can be activated as it rests within a reactor vessel.

Another aspect of the invention provides for use of activated catalyst materials produced by the present methods, in a reactor for synthesis of alcohols, such as ethanol. When alcohols are desired, suitable catalysts may include, but are not limited to, those disclosed in co-pending and commonly assigned U.S. patent application Ser. No. 12/166,167.

In some embodiments, as illustrating in the examples herein, the ethanol/methanol selectivity ratio is higher when activating a suitable promoted Co/Mo/S catalyst at lower pressure. The ethanol/methanol selectivity ratio can be tuned to be greater than unity, such as 1.1, 1.2, 1.3, 1.4, 1.5, or greater, by optimizing catalyst-activation conditions.

The carbon-atom selectivity ratio of ethanol to methanol is preferably higher than the carbon-atom selectivity ratio produced in a comparative method wherein the contacting (for catalyst activation) is carried out at an activation temperature outside the range of 200-350° C. and/or an activation pressure outside the range of 25-85 atm. This comparative method includes converting syngas to methanol and ethanol over the activated catalyst at a suitable process temperature and pressure to provide for substantially the same CO conversion. Such comparison can account for the dependence of methanol and ethanol selectivities on CO conversion, in the absence of catalyst-activation effects.

Preferred catalysts increase the rate of formation, selectivity, and/or yield of alcohols. Preferred catalysts also minimize the formation of CO₂ and CH₄ under reaction conditions that produce alcohols from syngas.

Other suitable catalysts to be activated by the present methods may include alkali/ZnO/Cr₂O₃, Cu/ZnO, Cu/ZnO/Al₂O₃, CuO/CoO, CuO/CoO/Al₂O₃, Co/S, Mo/S, Co/Mo/S, Ni/S, Ni/Mo/S, Ni/Co/Mo/S, Rh/Ti/SiO₂, Rh/Mn/SiO₂, Rh/Ti/Fe/Ir/SiO2, Rh/Mn/MCM-41, Cu, Zn, Rh, Ti, Fe, Ir, and mixtures thereof. The addition of basic promoters (e.g., K, Li, Na, Rb, Cs, and Fr) increases the activity and selectivity of some of these catalysts for ethanol or other C₂₊ alcohols. Basic promoters include alkaline-earth and rare-earth metals. Non-metallic bases can also serve as effective promoters, in some embodiments.

The reactor is any apparatus capable of being effective for producing at least one C₁-C₄ alcohol from the syngas stream fed. The reactor can be a single vessel or a plurality of vessels. The reactor contains at least one catalyst composition that tends to catalyze the conversion of syngas into alcohols. The “reactor” can actually be a series or network of several reactors in various arrangements. For example, in some variations, the reactor comprises a large number of tubes filled with one or more catalysts as provided herein.

The reactor for converting syngas into alcohols can be engineered and operated in a wide variety of ways. Operation that is substantially continuous and at steady state is preferable, but is not necessary to carry out the invention. The flow pattern can be substantially plug flow, substantially well-mixed, or a flow pattern between these extremes. The flow direction can be vertical-upflow, vertical-downflow, or horizontal. A vertical configuration can be preferable.

In some embodiments, fresh syngas is produced according to methods described in Klepper et al., “Methods and apparatus for producing syngas,” U.S. patent application Ser. No. 12/166,167 (filed Jul. 1, 2008), the assignee of which is the same as the assignee of the present application. U.S. patent application Ser. No. 12/166,167 is hereby incorporated by reference herein in its entirety.

In some embodiments, conditions effective for producing alcohols from syngas include reactor temperatures from about 200-400° C., preferably about 250-350° C. Depending on the catalyst chosen, changes to reactor temperature can change conversions, selectivities, and catalyst stability. As is recognized in the art, increasing temperatures can sometimes be used to compensate for reduced catalyst activity over long operating times.

Preferably, the syngas entering the reactor is compressed. Generally, catalyst productivity increases with increasing partial pressures of reactants. High reactor-inlet pressures realized by the presence of large quantities of unreactive gases are less preferable compared to higher partial pressures of the rate-limiting reactant(s). Conditions effective for producing alcohols from syngas include hydrogen and carbon monoxide partial pressures each about 10-200 atm or higher, preferably each about 25-100 atm. In some embodiments wherein H₂ is the rate-limiting reactant, it is beneficial to employ a higher partial pressure of H₂ than that of CO.

The input partial pressures define the feed hydrogen-carbon monoxide molar ratio (H₂/CO). While reaction rates are a function of species partial pressures, it can be a matter of convenience to specify H₂/CO for reasons of control and optimization within a certain process region. In some embodiments, conditions effective for producing alcohols from syngas include H₂/CO from about 0.2-4.0, preferably about 0.5-2.0, and more preferably about 0.5-1.5. These ratios are indicative of certain embodiments and are by no means limiting. It is possible to operate at feed H₂/CO ratios less than 0.2 as well as greater than 4, including 5, 10, or even higher.

In various embodiments, the feed to the reactor can include not only syngas but also one or more gases such as carbon dioxide, methane, ethane, ethylene, propane, propylene, methanol, ethanol, propanol, and higher hydrocarbons. The feed to the reactor can also include one or more inert or substantially inert gases.

In some embodiments, conditions effective for activation and conversion to alcohols from syngas include average reactor residence times from about 0.1-10 seconds, preferably about 0.5-2 seconds. “Average reactor residence time” is the mean of the residence-time distribution of the mobile-phase reactor contents under actual operating conditions. Catalyst contact times can also be calculated by a skilled artisan and these times will typically also be in the range of 0.1-10 seconds, although it will be appreciated that it is certainly possible to operate at shorter or longer times.

The catalyst phase can be a packed bed or a fluidized bed. The catalyst particles can be sized and configured such that the chemistry is, in some embodiments, mass-transfer-limited or kinetically limited. The catalyst can take the form of a powder, pellets, granules, beads, extrudates, and so on. When a catalyst support is optionally employed, the support may assume any physical form such as pellets, spheres, monolithic channels, etc. The supports may be coprecipitated with active metal species; or the support may be treated with the catalytic metal species and then used as is or formed into the aforementioned shapes; or the support may be formed into the aforementioned shapes and then treated with the catalytic species.

In general, the specific selection of catalyst configuration (geometry), temperature, partial pressures of both reactants and unreactive species, and residence time (or feed rate) will be selected to provide, or will be subject to constraints relating to, an economically optimized process. The plurality of reactor variables and other system parameters can be optimized, in whole or in part, by a variety of means.

Certain embodiments and aspects of the present invention will now be further described by way of the following examples.

### EXAMPLE 1

A catalyst is prepared wherein the catalyst composition comprises Co and Mo, combined with atomic ratio of Co to Mo of about 0.5. The catalyst composition also comprises sulfur, in an atomic ratio of S to (Co+Mo) of about 2. Potassium is introduced as K₂CO₃ so that the atomic ratio of K to (Co+Mo) is about 0.4. Thus 10 g of catalyst powder having a formula Co₁Mo₂S₆ is promoted by the addition of 1.9 g of K₂CO₃ (anhydrous). This catalyst composition is subjected to various experiments as described in the following examples.

### EXAMPLE 2

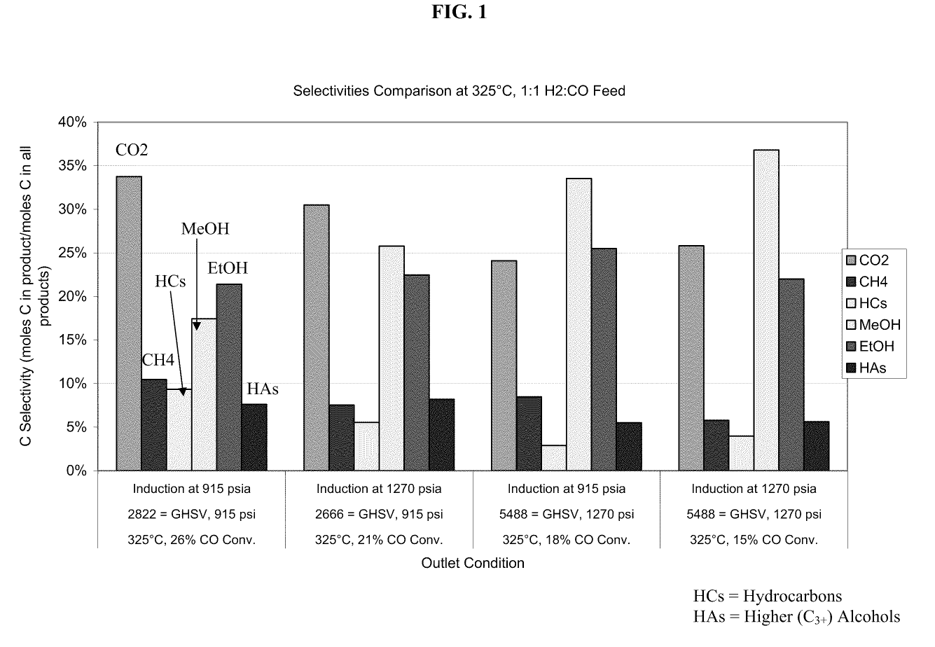

An experiment is carried out by first activating the starting catalyst in accordance with Example 1 at a pressure of 1270 psia in 1:1 H₂:CO. Then, the activated catalyst is evaluated at a pressure of 1270 psia at temperatures of 310° C. and 325° C. The activated catalyst is also evaluated at a pressure of 915 psia at temperatures of 310° C. and 325° C. It is observed at 325° C. (FIG. 1) that the carbon-atom selectivity to methanol is higher than that to ethanol.

### EXAMPLE 3

An experiment is carried out by first activating a starting catalyst in accordance with Example 1 at a pressure of 915 psia. The activated catalyst is then operated at pressure of 915 psia at temperatures of 310° C. and 325° C. This activated catalyst is also operated at a pressure of 1270 psia at temperatures of 310° C. and 325° C. The results are summarized in the chart shown in FIG. 1, for a temperature of 325° C. When the catalyst activated at 915 psia (“Induction at 915 psia”) is operated at 915 psia, higher C-atom selectivity to ethanol than to methanol is observed. It is hypothesized (without being limited to any particular explanation or theory) that higher ethanol/methanol ratios arise, at least in part, due to more-effective catalyst activation.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A method of activating a starting catalyst, said method comprising contacting said starting catalyst with a gas phase comprising syngas, under conditions comprising an activation temperature selected from about 200-350° C. and an activation pressure selected from about 25-85 atm, thereby producing an activated catalyst.

2. The method of claim 1, wherein said activation pressure is selected from about 50-75 atm.

3. The method of claim 2, wherein said activation pressure is selected from about 60-65 atm.

4. The method of any of claims 1-3, wherein said activation temperature is selected from about 250-325° C.

5. The method of claim 4, wherein said activation temperature is selected from about 275-300° C.

6. The method of claim 1, wherein said activated catalyst comprises cobalt, molybdenum, sulfur, and potassium.

7. The method of claim 1, further comprising converting syngas to at least one C₁-C₄ alcohol over said activated catalyst.

8. The method of claim 7, wherein said activating and said converting are conducted in the same vessel.

9. The method of claim 1, wherein said activation temperature is about the same as a temperature employed in said converting step.

10. The method of claim 1, wherein said activation temperature is lower than a temperature employed in said converting step.

11. The method of claim 1, wherein said activation pressure is about the same as a pressure employed in said converting step.

12. The method of claim 1, wherein said activation pressure is lower than a pressure employed in said converting step.

13. The method of claim 13, wherein said activation pressure is at least 10 atm lower than a pressure employed in said converting step.

14. The method of claim 14, wherein said activation pressure is at least 20 atm lower than a pressure employed in said converting step.

15. The method of claim 7, wherein said at least one C₁-C₄ alcohol includes ethanol.

16. The method of claim 15, wherein said at least one C₁-C₄ alcohol further includes methanol.

17. A method of producing methanol and ethanol from syngas, said method comprising:
(a) providing a starting catalyst composition;
(b) contacting said starting catalyst with a gas phase comprising syngas, at an activation temperature selected from about 200-350° C. and an activation pressure selected from about 25-85 atm, thereby producing an activated catalyst; and
(c) converting an amount of said syngas to at least some methanol and ethanol over said activated catalyst at an effective process temperature and pressure;
wherein the carbon-atom selectivity ratio of ethanol to methanol, calculated at a CO conversion in accordance with step (c), is at least 0.75.

18. The method of claim 17, wherein said carbon-atom selectivity ratio of ethanol to methanol is at least 1.

19. The method of claim 18, wherein said carbon-atom selectivity ratio of ethanol to methanol is at least 1.1.

20. The method of claim 19, wherein said carbon-atom selectivity ratio of ethanol to methanol is at least 1.2.

21. The method of claim 20, wherein said carbon-atom selectivity ratio of ethanol to methanol is about 1.25.

22. The method of claim 17, wherein said CO conversion is at least 25%.

23. The method of claim 17, wherein said carbon-atom selectivity ratio of ethanol to methanol is higher than the carbon-atom selectivity ratio produced in a comparative method wherein said contacting is carried out at an activation temperature outside the range of 200-350° C. and/or an activation pressure outside the range of 25-85 atm, wherein said comparative method includes converting an amount of said syngas to at least some methanol and ethanol over said activated catalyst at a suitable process temperature and pressure to provide for substantially the same CO conversion.

24. A method of activating a Co/Mo/S/K catalyst, said method comprising contacting said Co/Mo/S/K catalyst with a gas phase comprising syngas, at an activation temperature selected from about 275-300° C. and an activation pressure selected from about 60-65 atm, thereby producing an activated catalyst effective for conversion to methanol and ethanol when in the presence of syngas at suitable alcohol-synthesis conditions, wherein at said suitable alcohol-synthesis conditions, the carbon-atom selectivity ratio of ethanol to methanol is at least 1.2.
